(19) United States
(12) Patent Application Publication
(10) Pub. No.: US 2008/0300198 A1
(43) Pub. Date: Dec. 4, 2008

(54) Olive Compositions and Methods for Treating Inflammatory Conditions

(76) Inventors: Kathleen Matt, Gilbert, AZ (US); Catherine M. Bitler, Menlo Park, CA (US)

Correspondence Address: King & Spalding LLP, P.O. Box 889, Belmont, CA, 94002-0889, US

(21) Appl. No.: 11/659,861

(22) PCT Filed: Aug. 9, 2005
(86) PCT No.: PCT/US05/28179

§ 371 Date: Apr. 10, 2008

Related U.S. Application Data
(60) Provisional application No. 60/600,238, filed on Aug. 9, 2004.
(60) Provisional application No. 60/672,460, filed on Apr. 18, 2005.

(51) Int. Cl. A61K 31/7048; A61P 29/00

(52) U.S. Cl. 514/27

(57) ABSTRACT

A method of treating an inflammatory condition with a hydroxytyrosol-rich composition. Improvement is monitored as a reduction in the levels of a biochemical marker such as homocysteine or C-reactive protein. The composition may be administered in an amount and for a period sufficient to effect a drop in the level of the biochemical marker.

### BACKGROUND

Olive oil, the principal fat component of the Mediterranean diet, has been associated with a lower incidence of coronary heart disease (Owen et al., Eur. J. Cancer, 36:1235-1247, 2000b; Parthasarathy et al., PNAS USA, 87:3894-3898, 1990; Mattson and Grundy, J. Lipid Res, 26:194-202, 1985) and certain cancers (d'Amicis and Farchi, in: Advances in Nutrition and Cancer 2, Zappia et al., Eds., pp. 67-72, Kluwer Academic/Plenum Publishers, New York, 1999; Braga et al., Cancer, 82:448-453, 1998; Martin-Moreno et al., Int. J. Cancer, 58:774-780, 1994). Several laboratories have reported that the hydrolysis of the olive oil phenolic oleuropein and other family members lead to small phenolic components with strong chemoprotective activity (Owen et al., J. Can. Res. Clin. Onc, 125:S31, 2000a; Manna et al., FEBS Letters, 470:341-344, 2000). In particular, the olive oil phenolic hydroxytyrosol prevents low density lipoprotein (LDL) oxidation (Visioli and Galli, Nutr Rev, 56(5 Pt 1):142-147, 1998), platelet aggregation (Petroni et al., Thromb Res, 78:151-160, 1995), and inhibits 5- and 12-lipoxygenases (de la Puerta et al., Biochemical Pharmacology, 57:445-449, 1999; Kohyama et al., Biosci Biotechnol Biochem, 61:347-350, 1997). Hydroxytyrosol has also been found to exert an inhibitory effect on peroxynitrite dependent DNA base modification and tyrosine nitration (Deiana et al., Free Radic Biol Med, 26:762-769, 1999), and it counteracts cytotoxicity induced by reactive oxygen species in various human cellular systems (Manna et al., FEBS Letters, 470:341-344, 2000). The use of hydroxytyrosol and oleuropein, simple and polyphenols, respectively, obtained from olive oil have further been used for the treatment of skin damage (Perricone, U.S. Pat. No. 6,437,004). Studies evaluating bioavailability have shown that hydroxytyrosol is dose-dependently absorbed in humans following ingestion (Visioli et al., FEBS Letters, 468:159-160, 2000). Olive-derived phenols have also been described for treatment of inflammation or inflammation associated conditions (Crea, U.S. Patent Publication 2004/0039066 A1). Finally, a diet rich in olive oil and cooked vegetables may reduce the risk of rheumatoid arthritis (Linos et al., Am J Clin Nutr, 70(6):1077-1082, 1999).

### SUMMARY

In one aspect, a method of treating an inflammatory condition in a human subject is described. The method comprises (i) administering a hydroxytyrosol-rich composition to the subject, (ii) monitoring improvement in the subject according to a reduction in the subject's homocysteine levels, and (iv) continuing to administer the hydroxytyrosol-rich composition in an amount and for a period sufficient to effect a drop in homocysteine level of at least 7.5%. In an embodiment, the hydroxytyrosol-rich composition has a weight ratio of hydroxytyrosol to oleuropein between about 10:1 and about 100:1. In another embodiment, the hydroxytyrosol-rich composition is pure or substantially pure hydroxytyrosol.

In an embodiment, the hydroxytyrosol-rich composition is administered orally. In one embodiment, the hydroxytyrosol-rich composition is administered at a dosage effective to deliver between about 5.4 to 10.8 mg of total polyphenols daily. In another embodiment, the hydroxytyrosol-rich composition is administered at a dosage effective to deliver between about 2.5 to 5 mg of hydroxytyrosol daily.

Monitoring improvement may include monitoring the subject's plasma, serum and/or saliva levels of a biochemical marker. In one embodiment, the biochemical marker is homocysteine. In this embodiment, the hydroxytyrosol-rich composition may be administered at a dose and period of time until a decrease in homocysteine of at least about 12.5% relative to pre-treatment level is achieved. The inflammatory condition may be rheumatoid arthritis, wherein the hydroxytyrosol-rich composition is administered until a decrease in homocysteine of at least about 15% relative to pre-treatment level is achieved. In another embodiment, the hydroxytyrosol-rich composition is administered until a decrease in homocysteine of at least about 20% relative to pre-treatment level is achieved. In yet another embodiment, the hydroxytyrosol-rich composition is administered until homocysteine levels are normal or substantially normal.

In another embodiment, the inflammatory condition is a vascular-disease and the hydroxytyrosol-rich composition is administered until a decrease in homocysteine of at least about 20% relative to pre-treatment level is achieved.

In another aspect, a method for reducing homocysteine plasma levels in a person at risk of an inflammatory disease associated with elevated homocysteine levels is described. The method comprises (i) administering a hydroxytyrosol-rich composition to the person in an amount and for a period effective to reduce plasma homocysteine levels to within a normal range of homocysteine. In one embodiment, the hydroxytyrosol-rich composition has a weight ratio of hydroxytyrosol to oleuropein between about 10:1 and about 100:1. In another embodiment, the hydroxytyrosol-rich composition is pure or substantially pure hydroxytyrosol.

In an embodiment, the hydroxytyrosol-rich composition is administered orally. In one embodiment, the hydroxytyrosol-rich composition is administered at a dosage effective to deliver between about 2.5 to 5 mg of hydroxytyrosol daily.

The hydroxytyrosol-rich composition may be administered at a dose and period of time until a decrease in homocysteine of at least about 7.5% relative to pre-treatment level of homocysteine is achieved.

In yet another aspect, a method for reducing the risk of cardiovascular disease in a patient having elevated plasma homocysteine levels comprises administering a hydroxytyrosol-rich composition to the subject at a dose and for a period effective to reduce patient plasma homocysteine to within a normal range of homocysteine. In another embodiment, the hydroxytyrosol-rich composition is administered at a dose and period of time until a decrease in homocysteine of at least about 7.5% relative to pre-treatment level of homocysteine is achieved

The hydroxytyrosol-rich composition may have a weight ratio of hydroxytyrosol to oleuropein between about 10:1 and about 100:1.

In an embodiment, the hydroxytyrosol-rich composition is administered orally. In another embodiment, the hydroxytyrosol-rich composition is administered at a dosage effective to deliver between about 2.5 to 5 mg of hydroxytyrosol daily.

In yet another aspect, a method for reducing the risk of cardiovascular disease in a subject having elevated plasma C-reactive protein (CRP) levels comprises administering a hydroxytyrosol-rich composition to the subject at a dose and for a period effective to reduce patient CRP levels to within a normal range.

In another aspect, a method of identifying, from a population of human subjects having an elevated plasma homocysteine level related to an inflammatory condition, those responsive subjects who will show the greatest response to treatment by oral administration of a hydroxytyrosol-rich composition. The method comprises (i) administering the hydroxytyrosol-rich composition at a dose and for a period effective to substantially lower the plasma homocysteine level in a responsive subject, (ii) monitoring the subject's homocysteine level, and (iii) identifying the subject as a responsive subject if the subject's homocysteine level has decreased to within a normal range.

The monitoring may include monitoring the subject's serum, plasma, and/or saliva homocysteine level.

In a further aspect, a method of treating an inflammatory condition in a human subject is described comprising (i) administering a hydroxytyrosol-rich composition to the subject, (ii) monitoring improvement in the subject according to a reduction in the subject's C-reactive protein (CRP) levels, and (iii) continuing said administering in an amount and for a period sufficient to effect a drop in CRP level of at least 50%.

In an embodiment, the hydroxytyrosol-rich composition has a weight ratio of hydroxytyrosol to oleuropein between about 10:1 and about 100:1. In another embodiment, the hydroxytyrosol-rich composition is pure or substantially pure hydroxytyrosol.

In an embodiment, the hydroxytyrosol-rich composition is administered orally. In one embodiment, the hydroxytyrosol-rich composition is administered at a dosage effective to deliver between about 5.4 to 10.8 mg of total polyphenols daily. In another embodiment, the hydroxytyrosol-rich composition is administered at a dosage effective to deliver between about 2.5 to 5 mg of hydroxytyrosol daily.

Monitoring improvement may include monitoring the subject's plasma, serum and/or saliva levels of CRP. In an embodiment, the hydroxytyrosol-rich composition may be administered at a dose and period of time until a decrease in CRP of at least about 75% relative to pre-treatment level is achieved.

In an embodiment, the condition is rheumatoid arthritis.

### BRIEF DESCRIPTION OF THE FIGURES

FIG. 1 shows the structures of phenolic compounds and their precursors detected in olive oil: ligstroside (I); oleuropein glucoside (II); aglycone of ligstroside (III); aglycone of oleuropein glucoside (IV); dialdehydic form of ligstroside aglycone lacking a carboxymethyl group (V); dialdehydic form of oleuropein glucoside aglycone lacking a carboxymethyl group (VI); tyrosol (VII); hydroxytyrosol (VIII);

FIG. 2 is a graph of the POMS disturbance index;

FIG. 3 is a chart of the Physician Assessment of Disease for the placebo and the active ingredient at V2 and V5;

FIG. 4 is a graph of the diurnal cortisol levels of all patients at V1 and V5 as μg/dl cortisol over time;

FIG. 5 is a chart of CRP levels in mg/L for OA and RA patients receiving the placebo or the hydroxytyrosol-rich composition at V1 or V5;

FIG. 6 is a chart of homocysteine levels in μM/L for OA and RA patients receiving the placebo or the hydroxytyrosol-rich composition at V1 or V5;

FIG. 7 is a chart of the mean plasma values for MMP2, MMP3, or MMP9 in ng/ml±SE from V2 and V5 for patients receiving the placebo or the hydroxytyrosol-rich composition; and

FIG. 8 is a chart of the mean plasma values of TIMP in ng/ml±SE from V2 and V5 for OA and RA patients receiving the placebo or the hydroxytyrosol-rich composition.

### DETAILED DESCRIPTION

All publications, patents, patent applications or other references cited in this application are herein incorporated by reference in their entirety as if each individual publication, patent, patent application or reference are specifically and individually indicated to be incorporated by reference.

### I. Definitions

Unless otherwise indicated, all terms used herein have the same meaning as they would to one skilled in the art of the present invention. It is to be understood that this invention is not limited to the particular methodology, protocols, and reagents described, as these may vary.

The term “treatment” refers to inhibiting or arresting the development of a disease or condition in a patient, particularly a human, causing regression of the disease or condition, or relieving the symptoms associated with the disease or condition. The term “treating” includes prophylaxis of a physical condition or amelioration and/or elimination of the developed condition once it has been established or alleviation of the characteristic symptoms of such condition.

“Oral” refers to any route that involves administration by the mouth or direct administration into the stomach or intestines, including gastric administration.

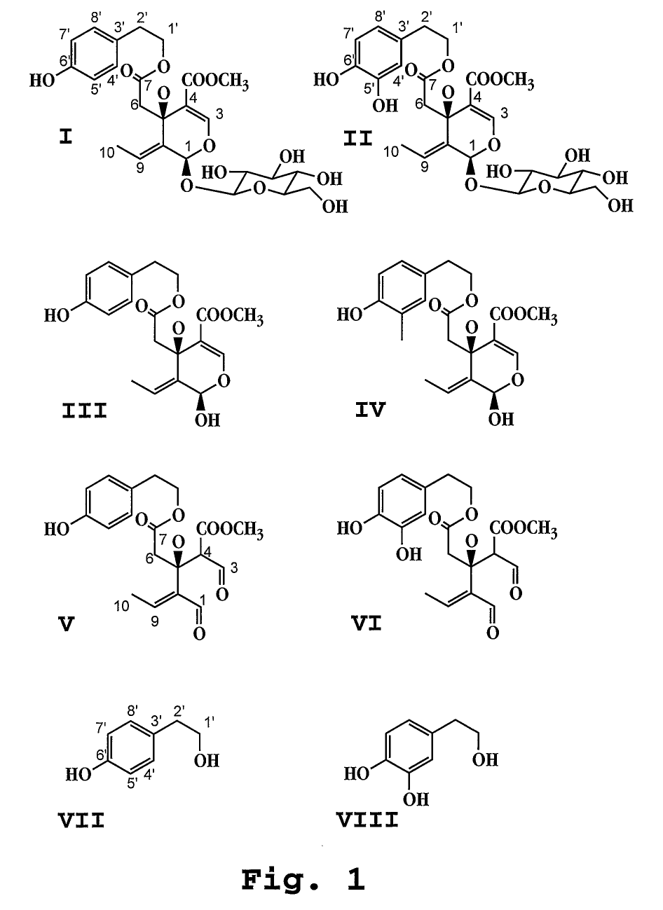

“Oleuropein” refers to secoiridoid glucoside oleuropein (Structure II in FIG. 1).

By “hydroxytyrosol” is intended 3,4-dihydroxyphenethyl alcohol (Structure VIII in the FIG. 1).

The term “effective amount”, as used herein, represents an amount of agent necessary to prevent or treat a subject susceptible to or suffering from an inflammatory response following administration to such subject. The term further represents an amount of a hydroxytyrosol-rich composition necessary to change plasma, serum, and/or salivary levels of a biochemical marker. The active compound may be effective over a wide dosage range. It will be understood that the amount of the compound actually administered will be determined by a physician, in light of the relevant circumstances including the condition to be treated the age, weight, and response of the individual patient, the severity of the patient's symptoms, and the chosen route of administration.

The term “substantially purified”, as used herein, refers to a compound or compounds that are removed from their natural environment, isolated or separated, and are at least 60% free, preferably 75% free, more preferably 85% free, even more preferably 90% free, still more preferably 95% free, and most preferably 99% free from other components with which they are naturally associated.

Abbreviations: IL6: interleukin-6; IL8: interleukin-8; IL2: interleukin-2; IL1β: interleukin-1β; MMP: matrix metalloproteinase; TIMP: Tissue inhibitor of metalloproteinase; HA: hyaluronan; COMP: cartilage oligomatrix protein; CRP: C-reactive protein; rheumatoid arthritis: RA; osteoarthritis: OA.

### II. Methods

A. Hydroxytyrosol-rich Compositions

In one aspect, the invention provides compositions effective to change plasma, serum, and/or saliva levels of a biochemical marker for treating inflammatory conditions. In another aspect, the invention provides compositions effective to change levels of a biochemical marker for reducing the risk of cardiovascular disease.

Hydroxytyrosol-rich compositions may be synthesized, extracted, and/or purified by methods known to those skilled in the art. In a preferred embodiment, the hydroxytyrosol-rich composition is obtained from vegetation water from olives.

1. Producing Hydroxytyrosol-rich Compositions from Vegetation Water

Hydroxytyrosol-rich compositions produced from the aqueous fraction of olive pulp, has bee described as a natural antioxidant with high levels of polyphenols (Quiles et al. 2002).

Conventionally, olive oil production involves crushing olives, including the pits, to produce a thick paste. During this procedure, the crushed olives are continuously washed with water, a process known as “malaxation.” The paste is then mechanically pressed to squeeze out the oil content. In addition to providing olive oil, the pressing also squeezes out the paste's water content. Such washing and pressing steps yield a considerable amount of water, referred to as “vegetation water.”

Both the pit and the pulp of olives are rich in water-soluble, phenolic compounds. Such compounds are extracted from olives during malaxation, according to their partition coefficients, and end up in the vegetation water. This explains why various phenolic compounds, such as oleuropein and its derivatives, produced in olive pulp, can be found in abundance in vegetation waters. Similarly, a number of monophenolic compounds, such as tyrosol and its derivatives, produced in olive pits, are also abundant in vegetation waters.

A hydroxytyrosol-rich composition from olive vegetation water may be prepared by adding acid to stabilize the vegetation water with the added benefit of preventing fermentation. In this manner, at least a portion of the oleuropein in the vegetation water is converted to hydroxytyrosol (Crea, U.S. Pat. No. 6,416,808 and related U.S. Publication No. 2003/0108651).

The olives may be obtained from conventional and/or commercially available sources such as growers. Preferably, the vegetation water is obtained from pitted olives. Pits in the olives contain tyrosol which is generally an undesired component in the vegetation water and which may not be appreciably broken down by the acid treatment described with reference to the hydroxytyrosol-rich composition described further below. The pits may be separated from the pulp manually or in an automated manner as described below. Preferably, such means should be capable of segregating the pits without breaking them, which might otherwise cause higher concentrations of tyrosol in the vegetation water. In another embodiment, the vegetation water is obtained from olives that have not been pitted.

To produce vegetation water, olive pulp from the olives is first pressed to obtain a liquid-phase mixture including olive oil, vegetation water, and solid by-products. Thereafter, the vegetation water is separated from the rest of the liquid phase mixture and collected. Exemplary methods of obtaining vegetation water are described in co-owned U.S. Pat. Nos. 6,165,475 and 6,197,308, both of which are expressly incorporated herein by reference in their entirety.

For purposes of commercial production, it may be desirable to automate various aspects of the invention. In this regard, one embodiment contemplates the use of an apparatus as disclosed in U.S. Pat. Nos. 4,452,744, 4,522,119 and 4,370,274, each to Finch et al., and each expressly incorporated herein by reference. Briefly, Finch et al. teach an apparatus for recovering olive oil from olives. Initially, olives are fed to a pulper that separates the olive pits from the olives to obtain a pitless olive meat. The meat is then taken up by an extraction screw that subjects the meat to an extraction pressure sufficient to withdraw a liquid phase, comprising oil, water and a minor proportion of olive pulp. The liquid phase is collected in a bin and then sent to a clarifying centrifuge that separates the pulp from the liquid phase to obtain a mixture comprising olive oil and vegetation water. A purifying centrifuge may be used to separate the vegetation water and a small proportion of solid matter from the mixture.

Additional devices that may be used in practicing the present invention are disclosed in Italian Patent Nos. 1276576 and 1278025, each of which is expressly incorporated herein by reference. As above, these devices can be used to separate the pulp from the pits prior to processing of the crushed olive pulp into oil, water, and solid residues.

As described above, the vegetation water is rich in water-soluble, phenolic compounds. Olive pulp extract contains about 6-9% total phenolic compounds. The structures of the phenolic compounds and their precursors detected in olive oil are shown in FIG. 1: ligstroside (I); oleuropein glucoside (II); aglycone of ligstroside (III); aglycone of oleuropein glucoside (IV); dialdehydic form of ligstroside aglycone lacking a carboxymethyl group (V); dialdehydic form of oleuropein glucoside aglycone lacking a carboxymethyl group (VI); tyrosol (VII); and hydroxytyrosol (VIII). Hydroxytyrosol typically comprises about 40-50% of the total phenolic compounds in the olive pulp solid extract. It will be appreciated that the composition may include one, several, or all of the phenolic compositions in varying ratios. It will further be appreciated that the vegetation water composition may be formulated to comprise a desired amount and/or ratio of any combination of the phenolic compounds.

Preferably, at least a portion of the oleuropein contained in the vegetation water is converted to hydroxytyrosol to prepare the hydroxytyrosol-rich composition. In one embodiment, as described in co-owned U.S. Pat. No. 6,416,808 and U.S. Application No. 2003/0108651, the pH of the vegetation water may be decreased by the addition of acid, and the vegetation water be allowed to incubate under conditions which promote acid hydrolysis of oleuropein to hydroxytyrosol. The sample may then be fractionated or extracted to separate hydroxytyrosol from other compounds.

The acid is added to the vegetation water preferably to adjust the pH between 1 and 5, and more preferably to adjust the pH between 2 and 4. Preferably, citric acid is used to adjust the pH of the vegetation water. Solid citric acid can be added while continuously stirring in an amount of about 10 to 20 kg of acid per about 1000 liters of vegetation water. The pH of the resulting solution can be monitored, and the pH adjusted accordingly such as by addition of more acid to achieve and maintain the desired pH.

In other embodiments, the acid may be an organic or inorganic acid other than citric acid. Exemplary acids include the inorganic substances known as the mineral acids, including sulfuric, nitric, hydrochloric, and phosphoric acids. Further exemplary acids are the organic compounds belonging to the carboxylic acid, sulfonic acid, and phenol (benzyl) groups.

In one embodiment, the mixture is allowed to incubate until hydroxytyrosol comprises about 75-90% of the total combination of oleuropein and hydroxytyrosol. In another embodiment, substantially all of the oleuropein in the original mixture is converted to hydroxytyrosol.

2. Purification

Following the conversion of oleuropein to hydroxytyrosol, the incubated vegetation water may be purified or fractionated by any suitable method known in the art. Exemplary methods of fractionation include partitioning with an organic solvent, such as ethyl acetate, chromatographic methods, including gel chromatography and high pressure liquid chromatography (HPLC), or liquid extraction with supercritical fluids such as carbon dioxide. In other embodiments, the supercritical fluid is selected from methane, ethane, propane, butane, isobutane, ethene, propene, hydrofluorocarbons, tetrafluoromethane, chlorodifluoromethane, dinitrogen monoxide, sulphur hexafluoride, ammonia, and methyl chloride. It will be appreciated that more than one supercritical fluid may be used in combination.

Prior to extraction with a supercritical fluid the vegetation water may have carriers such as maltodextran and/or polypropylene beads, added to the solution. Additional purification methods may also be used in accordance with the invention as mentioned above. HPLC isolation of hydroxytyrosol is described in Ficarra et al., Farmaco, 46:803-815, 1991; Romani et al., J Agric Food Chem, 47:964-967, 1999; and Tsimidou, Food Chem, 44:53-60,1992, each of which is expressly incorporated by reference herein.

In another embodiment, the solution may be dried prior or following extraction or purification of the desired polyphenol. The drying step preferably removes at least about 90%, more preferably at least about 95%, and even more preferably at least about 98% of the water from the vegetation water.

In one embodiment, vegetation water is obtained as described above and acidified to provide a solution which is rich in low molecular weight simple phenols and polyphenols, particularly hydroxytyrosol. In a preferred embodiment, the vegetation water is selectively enriched for hydroxytyrosol without the presence of other components. Thus, the major polyphenolic component, hydroxytyrosol, is isolated or enriched from other members of the polyphenolic family, impurities, suspended solids, tannins, and other molecules contained in the vegetation water.

In yet another embodiment, the composition is comprised of pure or substantially pure hydroxytyrosol.

B. Treatment

As described further below, in one aspect, the hydroxytyrosol-rich composition is useful in a method of treating an inflammatory condition in a human subject such that administration of the composition effects a change in plasma, serum, and/or saliva levels of one or more biochemical markers. Suitable biochemical markers include, but are not limited to cytokines, MMP, C-reactive protein, and/or homocysteine. It will be appreciated that the change in biochemical marker level may be a reduction or increase based on the marker. Measurements of plasma, serum and/or saliva levels may be as described further in Example 1 and/or by methods known in the art.

In one embodiment, the inflammatory condition is arthritis. Arthritis is a group of inflammatory conditions that affect the health of the bone joints in the body. One in five adults in the United States suffer from some form of arthritis (Vital Health Stat, 10(222), 2004). Recent statistics show 7.9% of persons aged 18-44 (8.5 million), 28.8% of persons aged 45-64 (18.5 million), and 47.8% of persons aged 65+ (15.7 million) report doctor-diagnosed arthritis (MMWR, 54(5):119-123, 2005).

There are over 100 types of arthritic diseases including rheumatoid arthritis (RA), osteoarthritis (OA), juvenile arthritis, psoriatic arthritis, Reiter's syndrome, and lupus. The cause of arthritic diseases is varied and includes autoimmune diseases such as rheumatoid arthritis and psoriatic arthritis; joint infection such as septic arthritis; the more common osteoarthritis, or degenerative joint disease. Although arthritis is primarily a disease that affects older individuals, it is not just an adult disease as approximately one in 1,000 children under the age of 16 suffers from arthritis (www.arthritis.ca). A common symptom in many arthritic diseases is inflammation of the joints.

Over the last decade there has been much interest in the anti-oxidative properties of polyphenols derived from olive oil. There is a substantial literature suggesting that polyphenols are beneficial in reducing the deleterious effects of oxygen free radicals on inflammatory and stress-related disorders. (Carluccio et al. 2003).

1. Rheumatoid Arthritis

Rheumatoid arthritis (RA) is an autoimmune disease in which the synovial membranes or tissues lining the joints become inflamed, called synovitis. Over time, this inflammation may destroy the joint tissues, leading to disability (www.webmd.com). Rheumatoid arthritis is generally considered one of the most serious and disabling types of arthritis. Rheumatoid arthritis affects women more often than men as 70% of rheumatoid arthritis occurs in females (www.arthritis.org) and frequently begins between the ages of 40 and 60 (www.webmd.com).

The cause of rheumatoid arthritis is not yet fully understood. There may be a genetic predisposition for developing rheumatoid arthritis; however it is likely that a bacterial infection, viral infection, or other foreign substance may trigger the immune response.

The abnormal immune response causes ongoing inflammation of the tissues lining the joint, a breakdown of cartilage, and loosening of the ligaments and tendons supporting the joint. Ongoing inflammation also causes the synovium to grow into a thick, abnormal tissue called pannus. These processes may result in destruction of the cartilage, the underlying bone surrounding the joint, ligaments, and tendons, and eventually lead to deformed joints.

No single laboratory test is used to diagnose rheumatoid arthritis. Instead, rheumatoid arthritis is usually diagnosed on the basis of symptoms and by eliminating other diseases that can cause similar symptoms (www.webmd.com).

2. Osteoarthritis

Osteoarthritis (OA) is a disease of the cartilage in joints. Osteoarthritis causes progressive breakdown of cartilage until the bones, usually separated by cartilage, rub against each other. This results in damage to the tissue and underlying bone, which causes the painful joint symptoms of osteoarthritis.

Osteoarthritis is the most common form of arthritis and is a major cause of pain and disability in older adults. It most often affects the joints of the fingers, hips, knees, feet, or spine. Osteoarthritis usually causes less inflammation than other types of arthritis, such as rheumatoid arthritis.

Osteoarthritis results from chemical changes in the cartilage that cause it to break down faster than it can be produced. In most cases, the cause of this cartilage breakdown is unknown. In some cases, secondary osteoarthritis may develop as a result of another condition (www.webmd.com).

There is no cure for osteoarthritis, although many people can manage their symptoms with medication and lifestyle changes. In a few people, osteoarthritis becomes severe enough to require surgery to replace or fuse the worn joint.

Rheumatoid arthritis and osteoarthritis have some common pathological features. Inflammation is the catalyst for the resulting cartilage and bone damage for both diseases although in the case of OA the inflammation is believed to be secondary to other initiating factors (Quiles et al. 2002, Carluccio et al. 2003, Leanne et al. 2002, Manna et al. 1997). It is well supported that inflammation triggers the over-expression of many proteolytic enzymes including the matrix metalloproteases (MMPs) whose substrates include many of the structural proteins of cartilage including collagen, proteoglycan and other non-collagenous proteins which make up the extracellular matrix (Kaneko et al. 2000, Sakito et al. 1995, Duff 1994). Numerous reports have shown that among other inflammatory compounds the proinflammatory cytokines, particularly IL-1β and TNF-α, play a key role in stimulating chondrocytes, synoviocytes and fibroblasts of the joints to up-regulate MMP expression and activity. Both OA and RA patients have increased levels of several MMPs and proinflammatory cytokines in the synovial fluid of affected joints. Elevated plasma levels of MMPs and cytokines have been reported in RA sufferers and to some extent in OA patients as well. Measurements of these compounds in the plasma and synovial fluid of arthritis patients have been examined as possible biological markers for use in diagnosis as well as prognosis of these diseases.

In one embodiment, the hydroxytyrosol-rich composition is useful in treating or ameliorating the symptoms of arthritis. In another embodiment, the hydroxytyrosol-rich composition is useful in reducing inflammation associated with arthritis.

Without being limited as to theory, it is hypothesized that the hydroxytyrosol-rich composition might act to decrease disease activity by: (1) directly decreasing pain and inflammation through inhibition of pro-inflammatory cytokines and through stimulation of anti-inflammatory cytokines; and/or (2) acting indirectly through changes in hormones, specifically by inducing changes in plasma levels of cortisol and prolactin.

As shown from the results presented in Example 1, the hydroxytyrosol-rich composition had a significant effect on decreasing disease activity, as measured by a decrease in biochemical markers. The biochemical marker level may be determined from plasma, serum and/or saliva by methods described herein and/or methods known in the art.

In one embodiment, the biochemical marker is homocysteine. Preferably, the hydroxytyrosol-rich composition is useful to lower plasma homocysteine levels by at least 7.5%, 12.5%, 15% or 20%. In another embodiment, the hydroxytyrosol-rich composition is useful to lower plasma homocysteine levels to at or below normal levels.

In another embodiment, the biochemical marker is C-reactive protein (CRP), a marker of inflammation. Preferably, the hydroxytyrosol-rich composition is useful to lower plasma CRP levels by at least 50%. In another embodiment, the hydroxytyrosol-rich composition is useful to lower plasma CRP levels by at least 63 or 65%. In another embodiment, the hydroxytyrosol-rich composition is useful to lower plasma CRP levels to at or below normal levels.

Often, the most powerful data, or the most relevant measure of effect of a supplement or treatment, is measured by changes in the HAQ (Health Assessment Questionnaire). The HAQ is used frequently as the outcome measure for clinical trials in rheumatoid arthritis and other diseases. The HAQ determines, through questionnaires, the degree of difficulty the patient experiences in performing physical functions of daily living. It includes data on dressing, eating, arising, walking, hygiene, grip and reach. It is one of the first self-report functional status (disability) measures, and is widely used to predict successful aging, development of risk factor models for osteoarthritis, and to examine mortality risks in rheumatoid arthritis patients.

As seen in Table 4, all patients taking the hydroxytyrosol-rich composition reported significant improvements in activities of daily living indicating improvements in mobility, etc. The data especially shows the changes in activities of daily living as indicated by the improvements in the HAQ score. As seen in Tables 5 and 6, the differences in HAQ scores between the hydroxytyrosol-rich group and the placebo group were compared by the percentages in each group that stayed the same, got worst, or showed improvement. These results show improvement in an amount (>0.22) that would be noticed by a physician and would result in a substantial change in a patient's life style, as established in the literature. Results indicate that 55% of people in the hydroxytyrosol-rich group reported that quality of life changes got better vs. 38% in the placebo group. Also, 37% of the hydroxytyrosol-rich group showed an improvement in an amount >0.22 whereas only 29% of the placebo group showed this change. This analysis shows the effectiveness of hydroxytyrosol-rich compositions in arthritis patients.

These decreases in HAQ score show a statistically significant (p<0.05) and clinically significant, >0.20 change in HAQ score. These changes are especially significant given that the dose of the hydroxytyrosol-rich composition was moderate at 4 capsules per day and the study itself was relatively short in duration at 2 months.

The results also show a statistically significant improvement (p=0.0085) in the subject's self-assessment in the hydroxytyrosol-rich treated group vs. placebo-treated group. As seen in Tables 5 and 6, differences in HAQ response were strongly dependent on the disease diagnosis (OA vs. RA). OA subjects had a 3.16× propensity for improvement in their HAQ score than RA subjects (p=0.047). OA patients showed statistically significant improvements in disease activity; moreover, by week 8, ˜40% of patients reported improvements of at least >75% and ˜90% of patients reported improvements of at least >20%.

Changes in a HAQ score of >20% are notable as an absolute change of 0.22 or more in the HAQ represents a change in disability that physicians note as clinically relevant. Further patients with RA perceive the change of 0.22 or more as a difference in functional status (Kosinski et al. 2000).

Improvement in HAQ scores with hydroxytyrosol-rich treated subjects was more evident in OA subjects than in RA subjects. This may be due to the fact that RA is an autoimmune disease, in contrast to OA, which is often a result of damage to a joint resulting in localized disease activity (inflammation) at the joint. If the hydroxytyrosol-compositions are a stimulator of immune function, it is possible that disease activity in the RA patient will not decrease, although the hydroxytyrosol-rich compositions have actions to decrease inflammation and pain. The results suggest that there might be a mechanism of action that involves more specific pathways in the joint related to inflammation, etc.

The Profile of Mood States (POMS) determines the present emotional state of the individual by measuring: anger, confusion, depression, fatigue, vigor and tension. Subjects are asked to rate their feelings from 0 (not at all) to 4 (extremely) on a random list of 60 words associated with the 6 areas. The results are tallied for a score in each of the 6 areas to determine mood state.

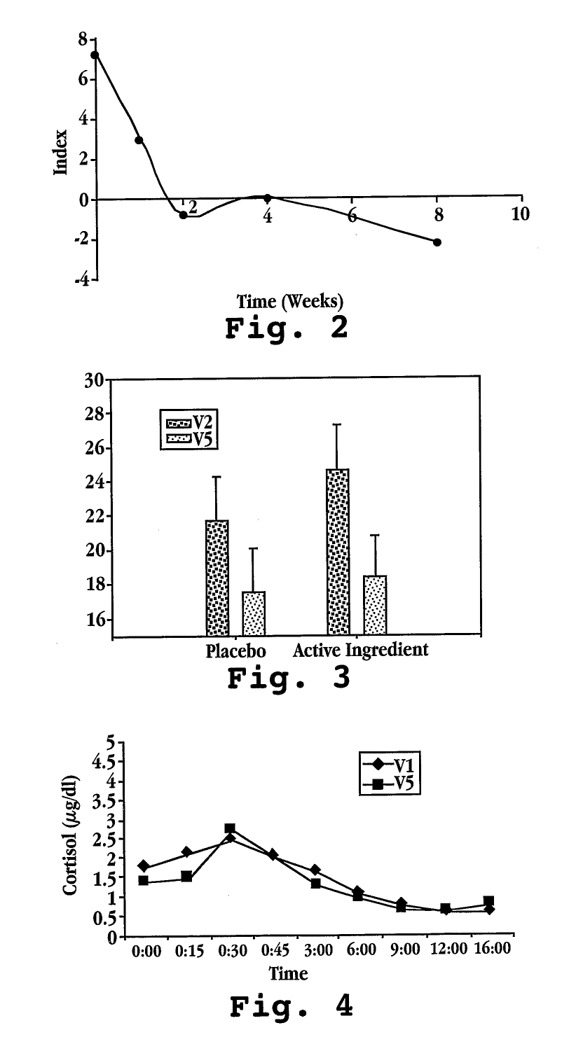

As seen in FIG. 2, a comparison of POMS for all subjects was done to assess the overall mood of the subjects from week to week. As shown below, initially all subjects had an unusually high disturbance score (about 7), which is a measurement of anxiety and tension. The score was reduced to about 3 at week 2 (steady state) and was at or below 0 for the rest of the study.

The high disturbance in subjects during the first visit was likely due to the newness of the environment as well as the uncertainty and anxiety of going to a new place, participating in a new research study with new people, new surroundings, etc. In order to control for this, subject data was compared between times V2 (after one week of treatment) and V5 (after 8 weeks of treatment) to obtain a measure of change in subjects that was not confounded by variations in mood. Results for three dependent variables were calculated based on a comparison of time V2 to time V5. For these comparisons, paired t-tests were used to evaluate changes between visit 2 (after 1 week) and visit 5 (after 8 weeks on supplement). By using the paired t-tests, changes over time were evaluated, using each individual as their own control. Given the number of variables that can affect disease activity such as severity of disease, medication, lifestyle, social support, diet, and exercise, using individuals as their own control, can help limit the impact of these variations.

Significant improvements (as measured by paired t-tests) in disease activity across time, as shown by two dependent measures (HAQ and Physician Assessment) were observed in the group taking the hydroxytyrosol-rich composition, but not in the group taking the placebo. As seen in FIG. 3, the Physician Assessment of disease decreased by at least 25% for patients taking the active ingredient as compared to about 18% for patients taking the placebo.

The results shown in Example 1 demonstrate that hydroxytyrosol-rich acts by some mechanism, as yet unidentified, to decrease pain and inflammation, and to increase mobility and activity in patients with osteoarthritis and rheumatoid arthritis.

FIG. 3 shows the scores for the physician assessment of disease at the second visit (V2) and the fifth visit (V5), with means+/−standard errors for individuals in placebo and control group. The assessment included evaluation of such factors as pain, motion and swelling of joints, the number of joints affected, and severity of arthritis. The paired t-tests comparing individual scores at time V2 vs. time V5 indicate significant decreases in the scores for the hydroxytyrosol-rich group (p<0.049), but no significant changes in the placebo group (with about 18% reduction in score). Both OA and RA patients are included in the analysis. This indicates that, as measured by physician assessment of disease activity, there is a significant decrease in the score from time 2 to time 5 in the hydroxytyrosol-rich group, indicating an effect of hydroxytyrosol-rich compositions to decrease disease activity as measured by the physician. The results show at least a 15% to 36% decrease in disease activity with further embodiments of 23-25% reduction in disease activity.

As seen in FIG. 4, a profile of the diurnal cortisol levels was similar between V1 (♦) and V5 (▪). Both profiles show increased cortisol for about 30 minutes from waking and decreased cortisol levels until about 9 hours from waking where cortisol levels stabilized at about 0.5-1 μg/dl.

As described above, in one embodiment, the biochemical marker is homocysteine whereby improvement in treating an inflammatory condition is monitored and effective to lower homocysteine levels. Homocysteine is a sulfur-containing amino acid that is not involved in the formation of proteins. Instead, it is an active component of two different metabolic pathways: the pathway involved in methionine formation and the pathway that converts cystathionine (a condensation product of homocysteine and serine) to cysteine and a-ketobutyrate.

As seen in FIG. 6, levels of homocysteine decreased significantly in rheumatoid arthritis patients in the group taking the hydroxytyrosol-rich composition, as seen by comparison of values, from visit 1 to visit 5 (p<0.015). In these studies, homocysteine levels significantly decreased in RA patients treated with the hydroxytyrosol-rich composition vs. RA patients treated with the placebo. As seen in the figure, homocysteine levels in RA patients decreased about 7.5% to 20% in the hydroxytyrosol-rich treatment group with embodiments of 12.5% and 15%. In one embodiment, the hydroxytyrosol-rich composition is effective to lower plasma and/or salivary levels to within a normal range. Although normal ranges may vary, 4-17 micromoles per liter (μmol/L) is generally considered within a normal range (www.webmd.com).

Elevated plasma levels of homocysteine have been associated with vascular disease, and homocysteine has been described as an independent risk factor for a variety of cardiovascular and cerebrovascular diseases. Further, elevated levels of homocysteine in the blood may promote plaque buildup in blood vessels, which increases the risk of atherosclerosis and subsequent coronary artery disease. Elevated homocysteine levels may also damage the lining of blood vessels, which may lead to the formation of blood clots; these, in turn, may increase the risk of stroke, heart attack (myocardial infarction), and pulmonary embolism. Also, increased homocysteine levels may promote the formation of blood clots in the deep veins of the legs (called deep venous thrombosis, or DVT). (www.webmd.com). It is thought that homocysteine has an oxidant stress effect on the vasculature that involves autoxidation of its sulfhydryl group generating superoxide radicals which in turn consume NO to form peroxynitrite. Accordingly, reducing plasma homocysteine levels reduces the risk of cardiovascular disease associated with elevated homocysteine levels.

In another embodiment, the biochemical marker is C-reactive protein (CRP). CRP is a member of the class of acute phase reactants where CRP levels increase with systemic inflammation. Measuring and charting CRP values have been used in determining disease progress or the effectiveness of treatments and CRP is considered a marker of inflammation. It has further recently been discovered that CRP plays a role in heart disease and that levels of CRP increase with cardiovascular risk (Abbate et al., Semin Vasc Med, 2003). It has additionally been shown that elevated CRP levels were associated with a threefold increase in the risk of a heart attack (Physicians Health Study clinical trial). Patients with elevated basal levels of CRP are at an increased risk for hypertension and cardiovascular disease. CRP levels relating to cardiovascular health are generally measured with the “high-sensitivity” CRP (hs-CRP) blood test. This is an automated blood test designed for greater accuracy in measuring low levels of CRP, which allows the physician to assess cardiovascular risk. The low-risk range is generally considered to be <1 mg/L (www.wikipedia.com). Normal CRP values vary by laboratory, but generally there is little or no CRP detectable in the blood (less than 0.6 mg/dL by the hs-CRP test) (www.nlm.nih.gov). Levels of CRP as determined by hs-CRP test between 1.0 and 3.0 mg/L is considered the average cardiovascular risk range. Levels above 3.0 mg/L (by hs-CRP) consider the patient to be at a high cardiovascular risk (www.americanheart.com). Normal levels of serum CRP as measured by a general CRP blood test are less than 10 mg/L. Most infections and inflammations result in CRP levels above 100 mg/L as measured by the CRP blood test. Accordingly, reducing plasma CRP levels reduces the risk of cardiovascular disease associated with elevated CRP levels.

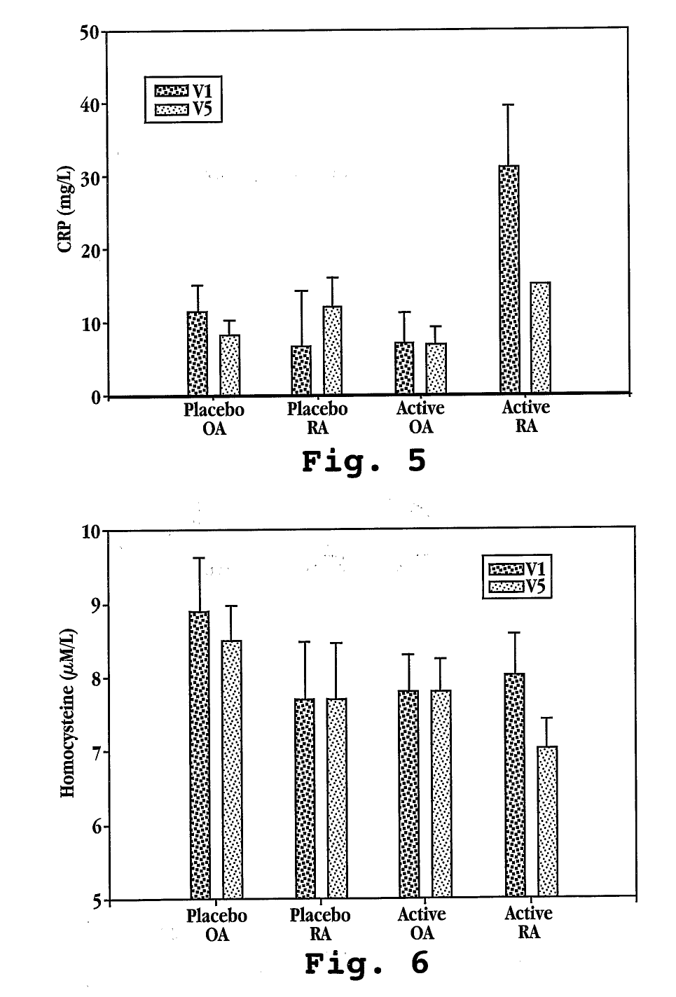

As described in Example 1, CRP levels significantly decreased in RA patients treated with the hydroxytyrosol-rich composition vs. RA patients treated with the placebo. As seen in FIG. 5, CRP levels in RA patients decreased about 50% to 63% in the hydroxytyrosol-rich treatment group. Analysis of the OA and RA subgroups of subjects indicates that the significant difference in CRP levels may be attributed to the hydroxytyrosol-rich composition. CRP levels initially highest in RA patients and in individuals with indications of extensive cardiovascular disease based on their medical records and their history of disease.

Changes in proinflammatory and anti-inflammatory cytokines, and metalloproteinases were further examined as possible biochemical markers as well as to identify a mechanism(s) of action of hydroxytyrosol-rich compositions. As seen in Tables 8 and 9, these markers were measured in a subgroup of the study participants. The data was examined and a group of subjects that had shown the most significant changes in HAQ were selected. These were determined to be the “responders” in the study and samples from these subjects were used to explore pro- and anti-inflammatory markers as a measure of hydroxytyrosol-rich composition action.

Cytokines were evaluated to determine the changes in the balance of pro- and anti-inflammatory cytokines. Cytokines were measured using either ELISA or the LUMINEX system as further described below. The values for the cytokines in the plasma were relatively low, but were higher in RA patients, as reflective of their underlying autoimmune disease. There were no significant changes in the cytokines comparing time 2 to time 5, using ANOVA, and as determined by paired t-test analysis (Table 9). However, the detection of changes in cytokines may be hampered by low levels in the plasma and to sensitivity limits of the assays.

Changes in plasma levels of cartilage oligomatrix protein (COMP) were evaluated since COMP has been shown in the literature to be related to inflammatory disease activity. COMP decreased somewhat in both the placebo and the hydroxytyrosol-rich groups, but not significantly in either group.

Plasma levels of HA were measured in order to determine possible mechanisms of action of hydroxytyrosol-rich compositions; however, as seen in Table 7, there was no indication that there was a significant effect or change from time 2 to 5.

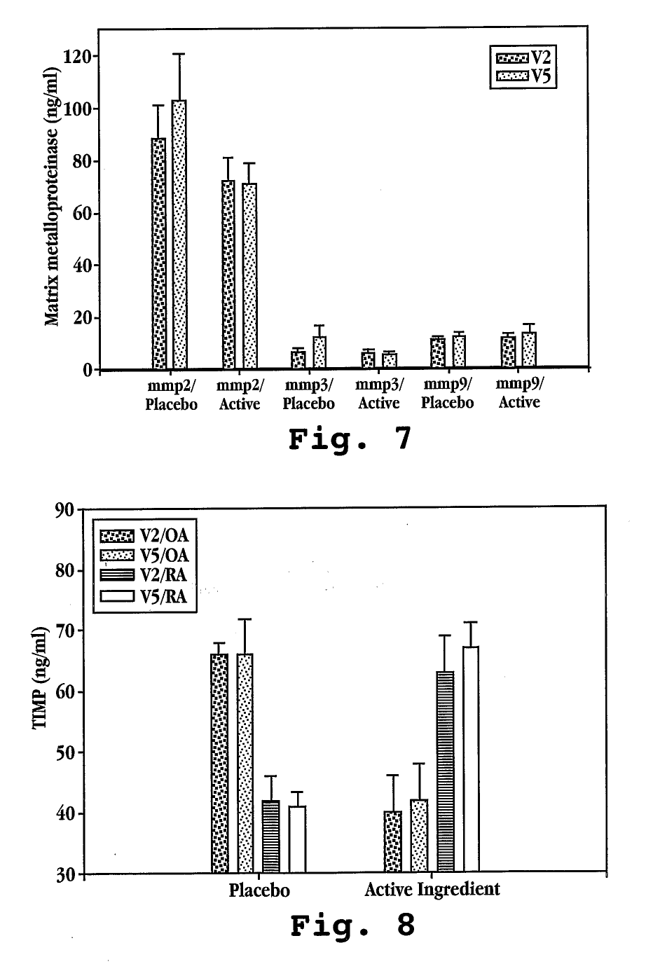

Matrix metalloproteinases or MMPs are Ca²⁺-activated zinc binding proteins that are secreted from cells in their latent, pro-enzyme form, and are involved in the degradation of a variety of matrix proteins. MMPs have been implicated in the pathogenesis of RA. Elevated levels of MMP3 and MMP1 are found in both the synovial fluid and serum of patients with RA. As described in Example 1, plasma levels of MMP2, MMP3 and MMP9 were evaluated by ELISA and the results shown in FIG. 7.

There was an increase in both MMP2 and MMP3 levels in the placebo-treated group that was not observed in the hydroxytyrosol-rich treated patients. In addition, in the placebo-treated group there was an increase in MMP1 in both OA patients and RA patients from time 2 to time 5. In the hydroxytyrosol-rich treated groups there was a decrease in MMP1 in OA patients and a slight increase in RA patients.

It should be noted that for MMP9, the levels observed were close to the sensitivity of the assay, and that the ELISA assay does not distinguish the pro- from the active form of the enzyme; thus, it cannot be determined from these results whether the patients have more or less of the active form, and thus are in a more pathogenic state.

Tissue Inhibitor of Metalloproteinase (TIMP) levels, which are the specific inhibitors of MMPs that only bind the active form, were also evaluated. Increased levels of TIMP-1 in the synovial fluid and serum of RA patients have been described in the literature (Giannelli, et al., Clin Exp Rheumatol, 2004, 22(3):335-338). However, as described, the ratio of TIMP to the active MMP enzyme appears to be critical for prediction of pathogenesis.

A slight decrease or no change in TIMP-1 levels was observed in the placebo-treated group and a slight increase in both OA patients and RA patients in the hydroxytyrosol-rich treated group (FIG. 8).

Although the above markers have generally been described with respect to plasma levels of the biomarkers, it will be appreciated that serum and/or salivary levels may be used as appropriate.

In another aspect, a method of identifying, from a population of human subjects having elevated levels of a biochemical marker related to an inflammatory or cardiovascular condition is contemplated. In this method, responsive subjects are considered to be those who will show the greatest response to treatment by administration of a hydroxytyrosol-rich composition. In a preferred embodiment, the hydroxytyrosol-rich composition is administered orally. The composition is administered at a dose and for a period effective to substantially lower the biochemical marker level in the subject. The biochemical marker level is monitored by methods known in the art and where the biochemical marker level is reduced, the subject is identified as responsive. In one embodiment, the biochemical marker level is reduced to within a normal range by administration of the biochemical marker. It will be appreciated that the method may be used to identify responsive subjects by effecting an increase of the biochemical marker level where appropriate. In a preferred embodiment, the biochemical marker is selected from homocysteine and/or CRP.

### IV. Administration

Routes of delivery include, but are not limited to, various systemic routes, including oral and parenteral routes (intravenous, subcutaneous, intraperitoneal, and intramuscular). Administration by these routes is achieved by formulating the compositions into a suitable dosage form. Non-limiting examples include pills, tablets, capsules, suspensions, syrups, liquid drops, and the like. Preparation of such dosage forms is routine to those of skill in the art. In a preferred embodiment, the composition is administered orally.

The composition may be administered either in substantially pure form (olive pulp solids or extract) or along with a pharmaceutically acceptable carrier. In one embodiment, the composition is dissolved or dispersed in the carrier as an active ingredient and formulated according to conventional practice. The carrier may be a solid form, semi-solid or liquid material which acts as a vehicle, carrier or medium for the active ingredient. Alternatively, the carrier can be in the form of a capsule or other container to facilitate oral administration. Thus, the oral dosage forms for administration in accordance with the present invention include tablets, pills, powders, capsules, syrups, liquids, and soft or hard gelatin capsules. The carrier may be any of a variety of standard physiologically acceptable carriers employed by those of ordinary skill in the art. It will be understood that the choice of suitable physiologically acceptable carrier will vary dependent upon the chosen mode of administration.

It will be appreciated that the hydroxytyrosol-rich composition can further be formulated to contain various weight ratios of the phenolic compounds. In one embodiment, the composition is formulated to contain various weight ratios of hydroxytyrosol to oleuropein. In preferred embodiments, the weight ratio of hydroxytyrosol to oleuropein is between 4:1 and 200:1, more preferably between about 10:1 and about 100:1. Preferred ratios of hydroxytyrosol to oleuropein include about 10:1 and about 100:1.

In another embodiment, the composition comprises purified hydroxytyrosol. In yet another embodiment, the composition comprises purified hydroxytyrosol in combination with a pharmaceutically suitable carrier. In a further embodiment, the composition comprises purified hydroxytyrosol administered in combination with other treatment compositions and methods.

The compositions for administration in the present invention may be formulated with other common pharmaceutically acceptable excipients as known in the art. Further, the compositions of the present invention may be formulated so as to provide quick, sustained or delayed release of the active ingredient after administration to a subject. Sustained or delayed release may be accomplished using any known method including semi-permeable polymeric matrices in the form of shaped articles such as films or microcapsules.

Parenteral formulations for use in accordance with the present invention are prepared using standard techniques in the art. The term parenteral as used herein includes subcutaneous injections, intravenous, intramuscular, intrasternal injection, or infusion techniques.

In one embodiment, the composition may be administered at regular intervals, e.g., daily, two times daily, or three times daily. In another embodiment, the composition is administered over a period of time, e.g. 1 to 12 months or more. It will be appreciated that administration of the composition may be continued for an indefinite time period. In yet another embodiment, the composition is administered for a period sufficient to effect a change in a biochemical marker indicating improvement in the inflammatory condition. As an example, the composition may be administered for one to two months or more. As a further example, the hydroxytyrosol-rich composition may be administered for a period of time sufficient to reduce plasma homocysteine levels by at least 7.5% or more and/or for a period of time sufficient to reduce plasma CRP levels by at least 50%. In another embodiment, the composition is administered for a period of time to reduce or increase biochemical marker levels to normal.

It will be appreciated that dosages of the composition will vary dependent upon the compound used in the composition. Preferred doses for oral administration of the composition include (i) from about 5-22 mg total simple phenols and polyphenols on a daily basis, with specific embodiments of 5, 5.4, 10, 10.8, 16, 16.2, 21.6, or 22 mg contemplated, and/or (ii) from about 2.5-5 mg or 2.5-10 mg hydroxytyrosol on a daily basis, with specific embodiments of 2.5, 5, 7.5, and 10 mg contemplated.

Dosages will vary in accordance with such factors as the age, health, sex, size and weight of the patient, the route of administration, and the efficacy of the compound. Greater or lesser amounts of the compound may be administered as required.

It will be appreciated that other components or active ingredients may be administered in combination with the treatment compound. It will further be appreciated that other treatment methods may be used in combination with administration of the treatment compound.

The following example illustrates methods of treating an inflammatory condition in a human subject comprising administering a hydroxytyrosol-rich composition to the subject and monitoring improvement in the subject according to a reduction in levels of biochemical markers. The composition was administered at an amount and for a period sufficient to effect a drop in biochemical marker levels. The examples are intended to illustrate, but in no way limit, the scope of the invention.

### V. Examples

A. Materials and Methods

Preparation of Treatment Composition

Capsules containing the active ingredient were formulated with a composition obtained from vegetation water from olives as in Table 1.

TABLE 1
Capsule formulation

| Ingredient | mg/capsule |
|---|---|
| olive pulp extract | 90 |
| (solids) | |
| citric acid | 15 |
| maltodextrin | 205 |
| silicon dioxide | 10 |
| magnesium | 10 |
| stearate | |
| modified cellulose | 60 |
| dicalcium | 600 |
| phosphate | |
| stearic acid | 70 |
| Total | 1060 |

Each capsule contained 90 mg of olive pulp extract (solids), 5.4 mg total polyphenols and ˜2.5 mg hydroxytyrosol.

Blood Sampling:

Blood was drawn, centrifuged at 3000 rpm for 30 minutes to separate plasma, and the plasma saved in numerous aliquots and stored at −50° C. Plasma samples were additionally centrifuged at 3,000 rpm for 15 minutes at 10° C. where recommended by the manufacturer (e.g. for MMPs and cytokine measurement protocols for Luminex). Plasma samples were aliquoted into microcentrifuge tubes and stored at −80° C. until assayed. Samples were allotted into measured aliquots to avoid multiple freeze thaw cycles which could interfere with accurate measurement of the samples, thus samples used for measurements were thawed only once.

The samples were measured for the following biochemical markers:

1. Matrix Metalloproteases (MMPs) and Cytokines

Plasma levels of MMPs (MMP-1, MMP-2, MMP-3, MMP-9 and MMP-13) and cytokines (IL-1β, IL-6, IL-8, IL-17, TNF-α, and GM-CSF) were measured using multianalyte profiling kits (R&D Systems, Minneapolis Minn. and Biosource International, Camarillo Calif.) in conjunction with the Luminex 100 analyzer. Briefly, this technique uses analyte-specific antibodies pre-coated on color coded beads. These microparticles were added to wells containing standards and samples. Following an incubation period and several washes analyte-specific biotinylated antibodies were added to the wells. The microparticles were washed again, followed by the addition of a streptavidin-phycoerythrin conjugate. The microparticles were washed and resuspended in buffer and analyzed on the Luminex 100 analyzer. The analyzer uses two lasers. The first determines which analyte is being measured and the second determines the magnitude of the fluorescent signal, which is directly proportional to the amount of analyte bound to the microparticle.

The MMP assay recognizes pro, mature and TIMP-1 complexed MMPs. The mean minimum detectable doses for MMPs were as follows: MMP-1: 4.4 pg/ml, MMP-2: 25.4 pg/ml, MMP-3: 1.3 pg/ml, MMP-9: 7.4 pg/ml and MMP-13: 159 pg/ml. The average intra-assay coefficient of variation (CV) for all MMPs was <10% and the inter-assay CV was <15%. The mean minimum detectable doses for cytokines were as follows: IL-1β: 0.27 pg/ml, IL-6: 0.36 pg/ml, IL-8: 3 pg/ml, TNF-α: 0.47 pg/ml, GM-CSF: 15 pg/ml, and IL-17: 0.39 pg/ml. The average intra assay CV for the R&D kit was <10% and for the BioSource Kits the average inter assay and intra assay CV was <10% and <15% respectively.

2. TIMP-1

Tissue inhibitor of metalloprotease-1 (TIMP-1) plasma levels were measured using a standard sandwich ELISA assay (R&D Systems) using the manufacturer's suggested protocol. This assay recognizes both natural and recombinant TIMP-1. The minimum detectable dose was 80 pg/ml and the inter-assay and intra-assay CVs were both <10%.

3. Hyaluronan

Hyaluronan (HA) was measured in plasma using a competitive ELISA assay (Echelon Biosciences Inc., Salt Lake City Utah) in which the colorimetric signal is inversely proportional to the concentration of HA in the sample. The assay recognizes intact as well as HA fragments. Inter-assay coefficient of variation was <15%. No minimum detectable dose was provided.

4. Cartilage Oligomatrix Protein

Cartilage oligomatrix protein (COMP) in the plasma was measured using an ELISA kit (AnaMar Medical Uppsala, Sweden) in which two monoclonal antibodies are directed against two antigenic determinants on the COMP molecule. The assay recognizes both intact and COMP fragments. The minimum detection limit was <0.1 U/L and the intra- and inter-assay CVs were both less than 10%.

5. C-Reactive Protein

Plasma levels of C-reactive protein (CRP) were measured using a high sensitivity ELISA assay (MP Biomedical, Orangeburg N.Y.). The manufacturer's protocol was adjusted by diluting all samples 1:500. The detectable range of the assay is approximately 0.01 mg/ml to 100 mg/ml. The intra-assay and inter-assay CVs were <15% and <10% respectively.

6. IL2R

Il-2 receptor antagonist levels were detected in plasma samples using a standard sandwich ELISA kit (R&D Systems). The protocol followed was that suggested by the manufacturer. The minimum detectable dose of the assay is <10 pg/ml. The inter-assay CV was <10%.

### EXAMPLE 1

100 patients aged 55-75 with a diagnosis of active RA or OA that were on a stable medication routine participated in a double-blind, randomized controlled trial. Measurements of disease activity, pain, inflammation, blood markers, cytokines, and other proteins, described further below, were measured in patients at baseline (time 0), week 1 (V2), week 2 (V3), week 4 (V4), and week 8 (V5), following initiation of the trial.

Fifteen subjects dropped out of the study before the last visit. A total of 85 subjects completed all five visits.

Of the 85 participants who completed all five visits, 44 patients (13 diagnosed with RA, 31 diagnosed with OA) received placebo capsules with inert contents. Additionally, 41 patients (13 diagnosed with RA, 28 diagnosed with OA) received capsules containing the active (hydroxytyrosol-rich) ingredient. The patients received two capsules twice per day of either active ingredient or placebo for a period of two months. Overall, the participants tolerated both the placebo and the hydroxytyrosol-rich composition well.

Measurements were obtained at baseline (time 0), at one week and two weeks to examine acute effects, and at four weeks and eight weeks to examine more chronic effects. Blood samples were measured for inflammatory markers, cytokines, blood chemistry, lipids, glucose, sedimentation rates, hemoglobin, and homocysteine levels. Specific levels were assessed for IL6, IL8, TNF, IL1B, IL2, MMP1, MMP3, MMP9, TIMP1, HA, and COMP.

Additionally, body composition was evaluated using a Dual-Energy X-Ray Absorptiometry (DEXA) scan and diurnal cortisol levels from salivary sampling were taken at baseline (time 0) and at the termination of the study (week 8):

A metabolic blood panel was taken at weeks 0 and 8 to assess any changes in liver and kidney function to assess safety.

TABLE 2
Patient Description

| | Placebo | Active Ingredient |
|---|---|---|
| | (n = 50) | (n = 49) |
| Age (years) | 64.4 ± 1.1 | 63.3 ± 1.0 |
| Weight (lbs) | 161.2 ± 4.6 | 181.8 ± 5.5 |
| BMI | 26.1 ± 0.6 | 29.6 ± 0.9 |
| % Fat | 37.1 ± 1.3 | 41.4 ± 1.3 |
| Heart Rate | 71.2 ± 1.4 | 69.8 ± 1.8 |
| Blood Pressure: | | |
| Systolic | 129.6 ± 2.5 | 132 ± 2.8 |
| Diastolic | 76.2 ± 1.3 | 78.0 ± 1.7 |
| Hematocrit (%) | 40.5 ± 0.6 | 40.7 ± 0.5 |
| Hemoglobin (g/dL) | 13.8 ± 0.2 | 14.0 ± 0.2 |
| No. of Smokers | 4 | 4 |
| No. on Glucosamine | 19 | 18 |

A summary of the medications taken by patients in the study is detailed in Table 3, below. All medication and supplement use were monitored throughout the study. Data indicates that by grouped medications there were no real differences in the medications between the placebo and active ingredient groups, although there were obviously differences between patients in the amount and type of their medications.

TABLE 3
Summary of Medications

| | # Patients | # Patients |
|---|---|---|
| | in placebo | in active |
| Medication | group | group |
| Narcotics and prescription analgesics | 3 | 8 |
| (Percoset, Vicodin ™, Ultram ™, | | |
| Tylenol ™ #3) | | |
| OTC analgesics (Tylenol ™, Aleve ™, | 9 | 13 |
| Advil ™, Bayer ™, and generics) | | |
| NSAIDS (Daypro ™, Vioxx ™, Celebrex ™, | 20 | 18 |
| Bextra, Naproxen, Voltaren ™, Relafen ™) | | |
| Antioxidants (Vitamins C, E, and B-12, | 17 | 14 |
| catechins containing supplements such | | |
| as tea extract) | | |
| Corticosteriods (Levothyroxine, | 3 | 3 |
| Albuterol, Flovent ™, Nasonex ™, | | |
| IM Cortisone) | | |
| Estrogen/Progesterone Replacement | 11 | 11 |
| (Estrodial, Premarin ™, Prempro) | | |
| Anti-depressants (Prozac ™, Clexa, | 3 | 6 |
| Amitriptyline, Serzone ™) | | |
| DMARS (Arava ™, Methotrexate, | 5 | 4 |
| Plaquenil ™) | | |
| Biological Response Modifiers | 0 | 1 |
| (Remicade ™) | | |
| Other supplements (multi-vitamins, | 35 | 32 |
| condrotin sulfate, herbal supplements) | | |
| Antibiotics (Tetracycline, Minocycline, | 2 | 1 |
| Augmentin ™) | | |
| Blood Thinner (Aspirin 81 mg) | 14 | 12 |
| Other hormones (Synthroid ™, Aurmour, | 8 | 6 |
| thyroid, Levoxyl ™, Levothroid ™) | | |
| Other Behavior Health Medications | 0 | 2 |
| (Clonazepam, Hydroxyzine, Lithiam, | | |
| Lamictal ™, Ativan ™) | | |
| Hypertensives (Tenormin ™, | 7 | 10 |
| Verapamil, Triamterene, | | |
| Norvasc ™, Hydrochlorthiazide) | | |
| Diabetic Medication (Amryl, | 2 | 1 |
| Glucophage ™) | | |
| Muscle Relaxants (Skelaxin ™, | 1 | 3 |
| Zanaflex ™) | | |
| Other (Cholesterol, acid suppressors, | 22 | 22 |
| sleep aids, etc) | | |

There were 3 adverse events which resulted in the participant having to discontinue taking capsules. One patient in each of the placebo and active ingredient groups had an allergic reaction. The supplement was stopped in each case and the subject dropped from the study. Also, one patient in the placebo group was dropped due to a sudden and painful flare of disease activity.

A. Assessment of Disease

Standard questionnaires designed to assess degree of disease-associated inflammation were administered by a physician, including the number of tender and swollen joints, the patient's rating of disease activity, and a physician rating of disease activity. Quality of life changes were measured by a Health Assessment Questionnaire (HAQ) with the results shown in Table 4, below. The separate results for the OA and RA patients are shown in Tables 5 and 6, respectively. The degree of mood changes was assessed by a Profile of Mood States (POMS) questionnaire. The disturbance score was plotted as a function of time with the results presented in FIG. 2. Initially all subjects had an unusually high disturbance score (about 7), which is a measurement of anxiety and tension. The disturbance score settled down to steady state levels by the third visit (week 2) (from about −2 to 0).

TABLE 4
HAQ quality of life changes

| | Placebo | Active ingredient |
|---|---|---|
| No change | 29% | 21% |
| Got worst | 33% | 24% |
| Got Better | 38% | 55% |
| Got Better than 0.22 | 29% | 37% |

TABLE 5
HAQ results for OA patients

| | Worse | | 0-25% improvement | | >25% improvement | |
|---|---|---|---|---|---|---|
| | | Active | | Active | | Active |
| Time | Placebo | Ingredient | Placebo | Ingredient | Placebo | Ingredient |
| Week 1 | 41% | 5.9% | 13% | 24% | 46% | 70% |
| Week 2 | 30% | 27% | 4.4% | 4.6% | 65% | 68% |
| Week 4 | 36% | 14% | 16% | 9.1% | 48% | 78% |
| Week 8 | 50% | 6.3% | 10% | 6.3% | 40% | 88% |

TABLE 6
HAQ results for RA patients

| | Worse | | 0-25% improvement | | >25% improvement | |
|---|---|---|---|---|---|---|
| | | Active | | Active | | Active |
| Time | Placebo | Ingredient | Placebo | Ingredient | Placebo | Ingredient |
| Week 1 | 11% | 0% | 22% | 75% | 66% | 39% |
| Week 2 | 33% | 7.7% | 17% | 38% | 50% | 47% |
| Week 4 | 30% | 9.1% | 20% | 27% | 50% | 63% |
| Week 8 | 20% | 0% | 30% | 43% | 50% | 57% |

Additionally, a Physician Assessment of Disease was established based on known measurements in the literature. The assessment consisted of: 1) a grading from 0-3 severe pain of each joint for tenderness and pain on motion and swelling of joints, 2) physician assessment of arthritis severity using a horizontal line to indicate severity, and 3) the physician used the patient's assessment of their disease as well as joint count to provide an overall assessment of disease presented in FIG. 3.

B. Biochemical Markers

Biochemical markers were measured from the plasma aliquots including MMP, cytokines, TIMP, hyaluronan, COMP, CRP and IL2R with the results presented in Table 7, below. Plasma levels for CRP in OA and RA patients are presented in Tables 8 and 9, respectively. Plasma levels of homocysteine in OA and RA patients are presented in Tables 10 and 11, respectively.

TABLE 7
Plasma levels for biochemical markers

| | Placebo | Placebo | Active | Active |
|---|---|---|---|---|
| | week 2 | week 8 | Ingredient | Ingredient |
| Biomarker | (V2) | (V5) | week 2 (V2) | week 8 (V5) |
| IL1B (pg/ml) | 39.2 | 29.1 | 31.1 | 30.1 |
| IL6 (pg/ml) | 29.8 | 30.7 | 27.9 | 29.5 |
| IL8 (pg/ml) | 13.3 | 11.9 | 12.4 | 19.5 |
| IL17 (pg/ml) | 7.6 | 6.7 | 6.7 | 6.7 |
| GM-CSF (pg/ml) | 50.2 | 45.8 | 57.2 | 52.8 |
| CRP (mg/L) | 8.9 | 15.9 | 9.7 | 11.28 |
| TNF (pg/ml) | 10.2 | 5.3 | 9.6 | 11.3 |
| COMP (U/L) | 19.2 | 11.4 | 21.5 | 18.4 |
| HA (ng/ml) | 140.7 | 163.4 | 134 | 140 |

TABLE 8
Plasma CRP levels (mg/L) for OA patients

| Visit | Placebo | Active ingredient |
|---|---|---|
| week 2 (V2) | 9.28 | 9.87 |
| week 8 (V5) | 7.52 | 9.75 |

TABLE 9
Plasma CRP levels (mg/L) for RA patients

| Visit | Placebo | Active ingredient |
|---|---|---|
| week 2 (V2) | 18.63 | 22.71 |
| week 8 (V5) | 21.11 | 17.23 |

TABLE 10
Plasma homocysteine levels (μM/L) for OA patients

| Visit | Placebo | Active ingredient |
|---|---|---|
| week 2 (V2) | 8.56 | 7.75 |
| week 8 (V5) | 8.58 | 6.03 |

TABLE 11
Plasma homocysteine levels (μM/L) for RA patients

| Visit | Placebo | Active ingredient |
|---|---|---|
| week 2 (V2) | 7.48 | 7.08 |
| week 8 (V5) | 7.95 | 7.02 |

C. Blood Chemistry

Blood lipids (total, triglycerides, HDL, LDL, and total cholesterol/HDL), sedimentation rates, blood glucose, homocysteine, hemoglobin (HGB), hematocrit (HCT), and erythrocyte sedimentation rate (ESR) were analyzed within 24 hours of blood draw by Sonora Qwest Laboratories with the results for OA and RA patients presented in Tables 12 and 13, respectively.

TABLE 12
Blood chemistry statistics for OA patients

| | | | Mean | Std. | Std. Error |
|---|---|---|---|---|---|
| | Group | N | Change | Deviation | Mean |
| Total Cholesterol | Placebo | 31 | 10.94 | 34.7 | 6.24 |
| | Active | 28 | 6.68 | 36.5 | 6.90 |
| Triglycerides | Placebo | 31 | 8.61 | 57.75 | 10.37 |
| | Active | 28 | −15.68 | 37.32 | 7.05 |
| HDL | Placebo | 31 | 2.39 | 11.05 | 1.98 |
| | Active | 28 | 2.64 | 7.62 | 1.44 |
| LDL | Placebo | 31 | 5.74 | 32.45 | 5.82 |
| | Active | 28 | 1.21 | 17.65 | 3.33 |
| Total | Placebo | 31 | .168 | .56 | .10 |
| cholesterol/HDL | Active | 28 | −2.05 | 10.28 | 1.94 |
| Glucose | Placebo | 31 | −1.65 | 7.35 | 1.32 |
| | Active | 28 | −.79 | 8.92 | 1.68 |
| Homocysteine | Placebo | 29 | .53 | 2.01 | .37 |
| | Active | 28 | −.07 | 1.22 | .23 |
| HCT | Placebo | 30 | .58 | 1.94 | .35 |
| | Active | 28 | .27 | 2.36 | .44 |
| HGB | Placebo | 31 | .22 | .60 | .10 |
| | Active | 28 | .20 | .67 | .12 |
| ESR | Placebo | 31 | .90 | 13.06 | 2.34 |
| | Active | 27 | 3.48 | 9.53 | 1.83 |

TABLE 13
Blood chemistry statistics for RA patients

| | | | Mean | Std. | Std. Error |
|---|---|---|---|---|---|
| | GROUP | N | Change | Deviation | Mean |
| Total Cholesterol | Placebo | 11 | 11.82 | 33.59 | 10.13 |
| | Active | 13 | 2.31 | 22.74 | 6.30 |
| Triglycerides | Placebo | 11 | −6.18 | 44.31 | 13.36 |
| | Active | 13 | −1.00 | 55.32 | 15.34 |
| HDL | Placebo | 11 | .45 | 12.56 | 3.78 |
| | Active | 13 | −2.31 | 9.98 | 2.77 |
| LDL | Placebo | 11 | 8.91 | 30.93 | 9.32 |
| | Active | 13 | 2.77 | 19.24 | 5.33 |
| Total | Placebo | 11 | −.018 | .570 | .17 |
| Cholesterol/HDL | Active | 13 | .069 | .537 | .14 |
| Glucose | Placebo | 11 | −1.36 | 5.35 | 1.61 |
| | Active | 13 | −2.85 | 10.90 | 3.02 |
| Homocysteine | Placebo | 11 | 5.92 | 19.33 | 5.83 |
| | Active | 13 | 1.00 | 1.27 | .35 |
| HCT | Placebo | 11 | 1.60 | 2.49 | .752 |
| | Active | 13 | 1.00 | 1.87 | .518 |
| HGB | Placebo | 11 | .55 | .77 | .23 |
| | Active | 13 | .29 | .46 | .12 |
| ESR | Placebo | 11 | −3.00 | 8.42 | 2.54 |
| | Active | 13 | .69 | 10.91 | 3.02 |

D. DEXA Scan Body Composition and Anthropometric Measures

Dual Energy X-ray Absorptiometry (DEXA) was used to measure body composition, including percent body fat and body fat distribution. Bone density was measured as well, but no changes in bone density were expected over this short time period. Total bone density was reported in g/cm² and as percentage of age-matched norms as seen in Table 14.

TABLE 14
Bone density (g/cm²)

| | % BF V1 | % BF V5 |
|---|---|---|
| Placebo | 37.70 | 38.00 |
| Active Ingredient | 42.6 | 41.35 |

E. Diurnal Salivary Cortisol

Diurnal Salivary Cortisol was measured with Salivettes (Sarstedt Inc., Rommelsdorf, Germany) at eight time periods, saliva sampling device, which consists of a small cotton swab inside a centrifugation tube. The sampling times were synchronized to the patient's awakening time. The first sample was taken immediately upon awakening and 3 samples taken at 15-minute intervals over the next 45 minutes. The last 4 samples were taken at 3 hour intervals, also synchronized to the awakening. The subjects did not to brush their teeth or ingest anything during the first 4 samples to avoid abrasion and vascular leakage and refrained from eating within 30 minutes for the rest of the sampling period. Patients did not exercise or exert themselves on the day of sampling (Edwards, et al., 2001). Once the samples were returned to the laboratory the Salivettes were centrifuged at 3000 rpm for 30 minutes at 4° C. and the saliva sample was transferred to a microcentrifuge tube and stored at −80° C. until time of analysis. Salivary cortisol was analyzed using a coated-tube RIA from commercially available kits (ICN Pharmaceuticals, Costa Mesa, Calif.). The results are shown in FIG. 4.

F. Salivary Cortisol Protocol

Saliva cortisol samples were collected from the subjects at two time points: a) after the first (i.e., baseline) visit but before the initiation of either placebo or supplement treatment, and b) one to two weeks prior to their final visit, after having been on either supplement or placebo for 1-2 months. Saliva samples were collected at nine time points on each sampling day as outlined in Table 15.

At each time point, the subjects placed a cotton swab from a Salivette sampling device (Sarstedt Ltd.) labeled with the appropriate sample number (1-9) into their mouth and chewed gently for 1-1.5 minutes. During the first four “awakening” samples (sample 1-4, Table 1) the subjects did not brush their teeth, eat breakfast, drink coffee, gargle, etc. during the 45 minutes of sampling to avoid micro-vascular leakage into the sample. The remaining samples were collected at 3 hour intervals, and subjects were instructed to avoid collection within 30 minutes of eating. In the laboratory, the samples were centrifuged, saliva collected and stored at −80° C. until assayed for cortisol.

TABLE 15
Sample times for collection of saliva for cortisol measurements

| Sample # | Expected Sample Time |
|---|---|
| 1 | 0:00 (Wake-up) |
| 2 | 0:15 |
| 3 | 0:30 |
| 4 | 0:45 |
| 5 | 3:00 |
| 6 | 6:00 |
| 7 | 9:00 |
| 8 | 12:00 |
| 9 | Bedtime |

The saliva samples were thawed and assayed for free cortisol using an ImmuChem Coated Tube Cortisol assay from MP Biomedicals (Costa Mesa, Calif.). The manufacturers suggested salivary procedure was followed with the slight modification of incubating samples for 24 hours at 5° C. prior to aspiration and counting with the results shown in Table 16.

Plasma prolactin was measured using an ImmuChem Coated Tube kit (MP Biomedicals, Costa Mesa, Calif.) with the results shown in Table 16. The procedure followed was that suggested by the manufacturer.

TABLE 16
Cortisol and Prolactin levels

| | | | Active | Active |
|---|---|---|---|---|
| | Placebo | Placebo | Ingredient | Ingredient |
| | V2 | V5 | V2 | V5 |
| Cortisol | 17.5 | 14.9 | 14.9 | 15.0 |
| Prolactin | 17.7 | 17.4 | 16.2 | 18.1 |

From the foregoing, it can be seen how various aspects and features of the invention are met. Those skilled in the art can now appreciate from the foregoing description that the broad teachings of the present invention can be implemented in a variety of forms. It will be appreciated that embodiments and subgroups described herein may be combined in the method and composition. Therefore, while this invention has been described in connection with particular embodiments and examples thereof, the true scope of the invention should not be so limited. Various changes and modification may be made without departing from the scope of the invention, as defined by the appended claims.

Although the invention has been described with respect to particular embodiments, it will be apparent to those skilled in the art that various changes and modifications can be made without departing from the invention.

It is claimed:

1. A method of treating an inflammatory condition in a human subject, comprising:
administering a hydroxytyrosol-rich composition to the subject,
monitoring improvement in the subject according to a reduction in the subject's homocysteine levels, and
continuing said administering in an amount and for a period sufficient to effect a drop in homocysteine level of at least 7.5%.

2. The method of claim 1, wherein said hydroxytyrosol-rich composition has a weight ratio of hydroxytyrosol to oleuropein between about 10:1 and about 100:1.

3. The method of claim 2, wherein said administering comprises oral administration, at a dosage effective to deliver between about 5.4 to 10.8 mg of total polyphenols daily.

4. The method of claim 2 wherein said administering comprises oral administration, at a dosage effective to deliver between about 2.5 to 5 mg of hydroxytyrosol daily.

5. The method of claim 1, wherein said monitoring includes monitoring the subject's plasma or saliva homocysteine level.

6. The method of claim 1, wherein said administering is continued until a decrease in homocysteine of at least about 12.5% relative to pre-treatment level is achieved.

7. The method of claim 1, for use in treating rheumatoid arthritis, wherein said administering is continued until a decrease in homocysteine of at least about 15% relative to pre-treatment level is achieved.

8. The method of claim 7, for use in treating rheumatoid arthritis, wherein said administering is continued until a decrease in homocysteine of at least about 20% relative to pre-treatment level is achieved.

9. The method of claim 1, for use in treating a vascular-disease inflammatory condition, wherein said administering is continued until a decrease in homocysteine of at least about 20% relative to pre-treatment level is achieved.

10. A method for reducing homocysteine plasma levels in a subject at risk of an inflammatory disease associated with elevated homocysteine levels, comprising:
administering a hydroxytyrosol-rich composition to the subject in an amount and for a period effective to reduce plasma homocysteine levels to within a normal range of homocysteine.

11. The method of claim 10, wherein said hydroxytyrosol-rich composition has a weight ratio of hydroxytyrosol to oleuropein between about 10:1 and about 100:1.

12. The method of claim 11, wherein said administering comprises oral administration, at a dosage effective to deliver between about 2.5 to 5 mg of hydroxytyrosol daily.

13. The method of claim 10, wherein said administering is continued until a decrease in homocysteine of at least about 7.5% relative to pre-treatment level is achieved.

14. A method for reducing the risk of cardiovascular disease in a patient having elevated plasma homocysteine levels, comprising:
administering a hydroxytyrosol-rich composition to the subject at a dose and for a period effective to reduce patient plasma homocysteine to within a normal range of homocysteine.

15. The method of claim 14, wherein said hydroxytyrosol-rich composition has a weight ratio of hydroxytyrosol to oleuropein between about 10:1 and about 100:1.

16. The method of claim 14, wherein said administering comprises oral administration, at a dosage effective to deliver between about 2.5 to 5 mg of hydroxytyrosol daily.

17. The method of claim 14, wherein said administering is continued until a decrease in homocysteine of at least about 7.5% relative to pre-treatment level is achieved.

18. A method for reducing the risk of cardiovascular disease in a subject having elevated plasma C-reactive protein (CRP) levels, comprising:
administering a hydroxytyrosol-rich composition to the subject at a dose and for a period effective to reduce patient CRP levels to within a normal range.

19. A method of identifying, from a population of human subjects having an elevated plasma homocysteine level related to an inflammatory condition, those responsive subjects who will show the greatest response to treatment by oral administration of a hydroxytyrosol-rich composition, comprising
administering the hydroxytyrosol-rich composition at a dose and for a period effective to substantially lower the plasma homocysteine level in a responsive subject,
monitoring the subject's homocysteine level, and
identifying the subject as a responsive subject if the subject's homocysteine level has decreased to within a normal range.

20. The method of claim 19, wherein said monitoring includes monitoring the subject's plasma or saliva homocysteine level.

21. A method of treating an inflammatory condition in a human subject, comprising:
administering a hydroxytyrosol-rich composition to the subject,
monitoring improvement in the subject according to a reduction in the subject's C-reactive protein (CRP) levels, and
continuing said administering in an amount and for a period sufficient to effect a drop in CRP level of at least 50%.

22. The method of claim 21, wherein said hydroxytyrosol-rich composition has a weight ratio of hydroxytyrosol to oleuropein between about 10:1 and about 100:1.

23. The method of claim 22, wherein said administering comprises oral administration, at a dosage effective to deliver between about 5.4 to 10.8 mg of total polyphenols daily.

24. The method of claim 22 wherein said administering comprises oral administration, at a dosage effective to deliver between about 2.5 to 5 mg of hydroxytyrosol daily.

25. The method of claim 22, wherein said decrease in CRP level is at least about 75% relative to pre-treatment level.

26. The method of claim 22, wherein said inflammatory condition is rheumatoid arthritis.
